(19) United States
(12) Patent Application Publication
(10) Pub. No.: US 2014/0348904 A1
(43) Pub. Date: Nov. 27, 2014

(54) Exosomes With Transferrin Peptides

(71) Applicant: Isis Innovation Limited, Oxford (GB)

(72) Inventors: Matthew Wood, Oxford Oxfordshire (GB); Samira Lakhal-Littleton, Oxford Oxfordshire (GB); Samir El Andaloussi, Oxford Oxfordshire (GB)

(21) Appl. No.: 14/363,677

(22) PCT Filed: Dec. 7, 2012
(86) PCT No.: PCT/GB2012/053053

§ 371 Date: Jun. 6, 2014

(30) Foreign Application Priority Data
Dec. 7, 2011 (GB) 1121069.7

(51) Int. Cl. A61K 38/40; C12N 15/113; C07K 14/47; A61K 47/48; C07K 14/79

(52) U.S. Cl. CPC A61K 38/40; A61K 47/48815; C07K 14/79; C07K 14/473; C12N 15/1137
USPC 424/450; 514/5.4; 424/184.1; 435/69.7; 536/23.4; 530/394; 435/320.1

(57) ABSTRACT

The present invention relates to exosomes comprising a transferrin targeting moiety on their surface, methods of producing them and to the use of such exosomes for delivering genetic material and/or biotherapeutic proteins or peptides or chemotherapeutic agents in vivo, in particular the use of such exosomes in methods of gene therapy or gene silencing, or delivery of other therapeutic agents.

### FIELD OF THE INVENTION

The present invention relates to the targeting of exosomes to a desired cell type or tissue. In particular the present invention relates to exosomes comprising a targeting moiety expressed on the surface of the exosome and methods of producing them.

### BACKGROUND TO THE INVENTION

Nucleic acids are routinely used in gene therapy for the replacement of non-functional genes [1] and for neutralization of disease-causing mutations via RNA interference (RNAi) effector molecules such as miRNAs [2], shRNAs [3] and siRNAs [4]. As naked DNA and RNA are difficult to deliver in vivo due to rapid clearance [5], nucleases [6], lack of organ-specific distribution and low efficacy of cellular uptake, specialized gene delivery vehicles are usually used for delivery.

Viral vectors and cationic liposomes are at the forefront of delivery vehicle technology and have been relatively successful with a large number of these delivery vehicles already in clinical trial [7]. Despite these successes, there remain significant limitations that restrict many applications, the most significant of which are immune recognition [8, 9, 10] for most viral vectors and mutagenic integration [11] for viruses such as lentiviruses; and inflammatory toxicity and rapid clearance for liposomes [12, 13, 14, 15]. Recognition by the innate immune system leads to acute inflammatory responses, which may require the use of immunosuppression strategies to overcome uptake and re-administration issues of current strategies [16, 17, 18] potentially exposing patients to unwarranted risks of opportunistic infections. Antibodies generated against the delivery vehicles also dramatically decrease transgene expression on subsequent administration [19].

The inherent risks and limitations of current strategies have generally limited them to life-threatening diseases of which the benefits of therapy clearly outweigh the risks, such as severe combined immunodeficiency [20], to diseases in special environments, such as immuno-privileged sites like the eye [1], or for genetic vaccination [21]. However, for genetic diseases which are chronic and debilitating but not life-threatening, such as myotonic dystrophy, a much lower risk profile and the ability to sustain corrective gene therapy for decades, not years, is required for curative intervention. An example of a potentially unacceptable risk for this class of diseases is immunosuppression strategies discussed above, highlighted by the death of a healthy patient in a recent AAV gene therapy trial for rheumatoid arthritis due to an opportunistic infection caused by immunosuppressants [22] taken by the subject unbeknownst to the trial administrators. With the increasing number of diseases shown to possess a genetic component, including obesity, heart disease and psychiatric illnesses, there is tremendous potential for the modification of susceptibility genes for preemptive genetic solutions, but only if the risks are further reduced and long-term sustainability is achieved. Hence, it is imperative to develop technologies that are able to avoid immune recognition and inflammation, while retaining good delivery efficiencies, in order to expand the use of gene therapy beyond lethal diseases.

One of the solutions may lie in the use of exosomes specifically targeted to a cell type or organ to be treated for gene delivery. Exosomes are small membrane-bound vesicles (30-100 nm) of endocytic origin that are released into the extracellular environment following fusion of multivesicular bodies with the plasma membrane. Exosome production has been described for many immune cells including B cells, T cells, and dendritic cells (DCs). Exosomes derived from B lymphocytes and mature DCs express MHC-II, MHC-I, CD86 and ICAM-1 [23, 24], and have been used to induce specific anti-tumor T cytotoxic responses and anti-tumor immunity in experimental models and clinical trials [24, 25]. The potential of exosome-mediated gene delivery has been shown with delivery of murine mRNAs and miRNAs to human mast cells [26] and glioma-derived exosomes [27] have been demonstrated to transfer mRNAs produced by exogenous DNA plasmids to heterologous cells. Exosomes with exogenous DNA, siRNAs and other modified oligonucleotides have also been disclosed [28].

### SUMMARY OF THE INVENTION

The present inventors have also successfully introduced targeting moieties into exosomes so that the exosomes can be targeted to a selected tissue. In particular, the present inventors have successfully introduced transferrin (Tf) peptide targeting moieties into exosomes so that the exosomes can be targeted to tissues or cells expressing the transferrin receptor (TfR). The present inventors have shown that exosomes expressing Tf peptide targeting moieties have comparable uptake efficiency with commerically available conventional transfection reagents such as lipofectamine and RNAi max. Thus the present invention relates to exosome comprising a Tf peptide targeting moiety on the surface, fusion proteins comprising exosomal transmembrane proteins and a targeting moiety, and to nucleic acid constructs encoding such fusion proteins. The Tf targeted exosomes may be loaded with cargo such as exogenous genetic material or biotherapeutic protein and/or peptide, and/or chemotherapeutic agents and used in the delivery of cargo, particularly the delivery of nucleic acids for gene therapy and gene silencing, and delivery of biotherapeutic proteins and peptides and chemotherapeutic agents for therapy.

In accordance with one aspect of the present invention, there is provided a composition comprising an exosome, wherein the exosome comprises a targeting moiety expressed on the surface of the exosome, and said targeting moiety is a transferrin (Tf) polypeptide or peptide. The Tf polypeptide or Tf peptide targeting moiety may be any Tf polypeptide or Tf peptide that retains the ability to bind to the TfR.

In another aspect, the invention provides a composition comprising an exosome which comprises a Tf polypeptide or a Tf peptide targeting moiety expressed on the surface of the exosome, wherein the exosome is derived from an immature dendritic cell, for use in a method of delivering the genetic material, protein and/or peptide in vivo.

In another aspect, the invention provides a method of producing an exosome comprising a Tf polypeptide or Tf peptide targeting moiety expressed on the surface of the exosome comprising expressing a fusion protein comprising the targeting moiety and an exosomal transmembrane protein within a cell used to produce exosomes, wherein the expressed fusion protein is incorporated into the exosome as it is produced by the cell.

In another aspect, the invention provides a polypeptide comprising an exosomal transmembrane protein, and a heterologous targeting protein, polypeptide or peptide, wherein the targeting protein, polypeptide or peptide is Tf polypeptide or a Tf pepetide that binds to TfR present on the surface of the cell to be targeted, and wherein when the polypeptide is present in an exosome, the targeting Tf polypeptide or Tf peptide is present on the surface of the exosome.

In another embodiment, the invention provides a polynucleotide construct encoding a polypeptide, wherein the construct comprises polynucleotide encoding a 5′ exosomal transmembrane signal sequence, for example an endoplasmic reticulum-targeting signal peptide operatively linked to polynucleotide encoding the polypeptide of the invention.

In another embodiment, the invention provides a method of targeting an exosome to a selected tissue or cell type comprising transfecting a host cell with a polynucleotide construct according to the invention, expressing the construct in the host cell, and obtaining exosomes from the host cell in which the construct has been expressed.

### DESCRIPTION OF THE FIGURES

FIG. 1. Knockdown of GAPDH with exosome-mediated siRNA delivery: (a, b) Representative images of Cy5-labelled GAPDH siRNA applied on to HEK cells with electroporated (E) untargeted (Exo untargeted), Tf-targeted (Exo Tf) or RVG (Exo RVG) targeted exosomes. Unelectroporated exosomes and siRNA (NE) were added as negative controls and siRNA and conventional transfection reagents (LIPO and RNAimax) were added as positive controls.

FIG. 2. Knockdown of GAPDH with exosome-mediated siRNA delivery: (a, b) Representative images of Cy5-labelled GAPDH siRNA applied on to B2M17 cells with electroporated (E) untargeted (Exo untargeted), Tf-targeted (Exo Tf) or RVG (Exo RVG) targeted exosomes. Unelectroporated exosomes and siRNA (NE) were added as negative controls and siRNA and conventional transfection reagents (LIPO and RNAimax) were added as positive controls.

FIG. 3. Knockdown of GAPDH with exosome-mediated siRNA delivery: (a, b) Representative images of Cy5-labelled GAPDH siRNA applied on to Neuro2a cells with electroporated (E) untargeted (Exo untargeted), Tf-targeted (Exo Tf) or RVG (Exo RVG) targeted exosomes. Unelectroporated exosomes and siRNA (NE) were added as negative controls and siRNA and conventional transfection reagents (LIPO and RNAimax) were added as positive controls.

FIG. 4. Knockdown of GAPDH with exosome-mediated siRNA delivery: (a, b) Representative images of Cy5-labelled GAPDH siRNA applied on to mouse bone marrow dendritic cells with electroporated (E) untargeted (Exo untargeted), Tf-targeted (Exo Tf) or RVG (Exo RVG) targeted exosomes. Unelectroporated exosomes and siRNA (NE) were added as negative controls and siRNA and conventional transfection reagents (LIPO and RNAimax) were added as positive controls.

### DETAILED DESCRIPTION OF THE INVENTION

The present invention is directed to exosomes, and their use as delivery vehicles. Exosomes are small membrane-bound vesicles of endocytic origin that are released into the extracellular environment following fusion of multivesicular bodies with the plasma membrane. Thus, the present application is directed to a composition comprising such exosomes. Typically, the exosomes are between 30 and 100 nm in diameter but can include membrane particles of similar origin up to 200 nm. Exosomes as used herein refers to nanoparticles of endosomal origin that are secreted from multivesicular bodies.

Exosomes are produced by many different types of cells including immune cells such as B lymphocytes, T lymphocytes, dendritic cells (DCs) and mast cells. Exosomes are also produced, for example, by glioma cells, platelets, reticulocytes, neurons, intestinal epithelial cells, tumour cells, HELA cells, human embryonic kidney cells (HEK cells), B2M17 cells, Bend3 cells, primary bone marrow-derived dendritic cells, BV-2 microglia cells and NEURO2A cells. Exosomes for use in accordance with the present application can be derived from any suitable cell, including the cells identified above. Exosomes have also been isolated from physiological fluids, such as plasma, urine, amniotic fluid and malignant effusions.

In a preferred aspect of the present invention, exosomes are derived from DCs, preferably immature DCs. Exosomes produced from immature DCs do not express MHC-II, MHC-I or CD86. As such, such exosomes do not stimulate naïve T cells to a significant extent and are unable to induce a response in a mixed lymphocyte reaction. Thus exosomes produced from immature dendritic cells are ideal candidates for use in delivery of cargo such as genetic material, particularly for in vivo use, for example, in gene therapy.

Thus, in accordance with the one aspect of the present invention, exosomes derived from dendritic cells are provided for use in in vivo targeted delivery of cargo such as genetic material and protein and/or peptide biotherapeutics, or chemotherapeutic agents. In a preferred embodiment, the exosomes of the invention are derived from dendritic cells and provided for use in in vivo targeted delivery of genetic material, protein or peptide biotherapeutics or chemotherapeutic agents.

As outlined above, exosomes are produced by many different types of cell and have also been isolated from physiological fluids. Thus, in accordance with the present invention, exosomes can be obtained from any suitable cell type as discussed above, or by isolation from physiological fluids. Typically, the methods of the present invention comprise isolation of the exosomes from cell culture medium or tissue supernatant.

Exosomes produced from cells can be collected from the culture medium by any suitable method. Typically a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 μm filter), gradient ultracentrifugation (for example, with sucrose gradient) or a combination of these methods.

In accordance with the present invention, the exosomes of the invention may be loaded with exogenous genetic material, protein or peptide, or with chemotherapeutic agents. In particular, in accordance with the present invention, exosomes can be prepared and then loaded with the desired genetic material, protein or peptide, or chemotherapeutic agent for targeted delivery. The exosomes can also be loaded with exogenous genetic material or with biotherapeutic proteins or peptides, by over expressing suitable constructs encoding the genetic material or protein or peptide in a cell, so that the material is loaded into the exosomes.

The use of Tf-targeted exosomes to deliver exogenous genetic material, protein or peptide offers a number of advantages over conventional means of delivering such molecules. For example, when compounds are delivered using exosomes they are protected from degradation and are more stable; compounds may be delivered to a target tissue, such as a specific type of cancer, more efficiently and/or more specifically than if not encapsulated inside exosomes; and/or the cargo may be less likely to elicit an immune response because it is contained within exosomes and so is not freely available for detection by immune cells and/or binding to antibodies. Other potential advantages of the use of Tf-targeted exosomes to deliver exogenous genetic material, protein or peptide or chemotherapeutic agent include bypassing hepatic cells following systemic delivery of exosomes and/or avoiding drug resistance, such as the upregulation of drug transporters such as ABC-transporters.

Suitable genetic material for delivery includes exogenous DNA plasmid and siRNA. Suitable genetic material also includes modified oligonucleotides and other RNA interference effector moieties such as miRNA and shRNA. In accordance with one aspect of the present invention, the genetic material loaded into the exosome is not genetic material that is typically associated with the exosomes, for example, the nucleic acid is preferably not an mRNA or a miRNA which is incorporated into an exosome on its production from a cell. In particular, the genetic material may be loaded into an exosome preparation that has already been isolated from cells. Alternatively the genetic material may be overexpressed in a cell, for example by introducing a suitable construct into the cell, and subsequently loaded into exosomes. Thus exogenous genetic material refers to genetic material inclusive of nucleic acids that is not normally associated with exosomes. In particularly preferred embodiments, the nucleic acid material is plasmid DNA or other nucleic acids such as siRNA and modified oligonucleotides which are not typically found in exosomes.

Similarly, preferably the protein or peptide loaded into the exosome is not protein or peptide that is typically associated with the exosomes, for example, the protein or peptide is preferably not a protein or peptide which is incorporated into an exosome on its production from a cell, such as an unmodified cell. Exogenous protein or peptide refers to proteins or peptides inclusive of protein or peptide that is not normally associated with exosomes. Such exogenous proteins or peptides can be loaded into an exosome which has been isolated from a cell, or by overexpression of a suitable construct in a host cell, such that the protein or peptide is loaded into the exosome. In particularly preferred embodiments, the proteins or peptides to be loaded into the exosomes are antibodies or antibody fragments, more preferably monoclonal antibodies or fragments thereof.

Nucleic acids for incorporation into the exosomes may be single or double stranded. Single-stranded nucleic acids include those with phosphodiester, 2′O-methyl, 2′ methoxy-ethyl, phosphoramidate, methylphosphonate, and/or phosphorothioate backbone chemistry. Typically double-stranded nucleic acids are introduced including for example plasmid DNA and small interfering RNAs (siRNAs).

Any biotherapeutic protein and/or peptide that has utility in the treatment and/or prevention of a condition, disease or disorder may be incorporated into exosomes according to the present invention. In a preferred embodiment the protein or peptide for incorporation into the exosomes is an antibody or an antibody fragment. In a more preferred embodiment, the antibody is a monoclonal antibody or fragment thereof.

Exosomes can also be loaded with chemotherapeutic agents, such as therapeutic pharmaceutical agents, for delivery of the chemotherapeutic agent. Examples of suitable agents include cytotoxic agents, for example for use in the treatment of cancer.

The genetic material, protein or peptide to be loaded into the exosomes is chosen on the basis of the desired effect of that genetic material, protein or peptide on the cell into which it is intended to be delivered and the mechanism by which that effect is to be carried out. For example, the nucleic acid may be useful in gene therapy, for example in order to express a desired gene in a cell or group of cells. Such nucleic acid is typically in the form of plasmid DNA or viral vector encoding the desired gene and operatively linked to appropriate regulatory sequences such as promoters, enhancers and the like such that the plasmid DNA is expressed once it has been delivered to the cells to be treated. Examples of diseases susceptible to gene therapy include haemophilia B (Factor IX), cystic fibrosis (CTFR) and spinal muscular atrophy (SMN-1).

Nucleic acid can also be used for example in immunisation to express one or more antigens against which it is desired to produce an immune response. Thus, the nucleic acid to be loaded into the exosome can encode one or more antigens against which is desired to produce an immune response, including but not limited to tumour antigens, antigens from pathogens such as viral, bacterial or fungal pathogens.

Nucleic acid can also be used in gene silencing. Such gene silencing may be useful in therapy to switch off aberrant gene expression or in animal model studies to create single or more genetic knock outs. Typically such nucleic acid is provided in the form of siRNAs. For example, RNAi molecules including siRNAs can be used to knock down DMPK with multiple CUG repeats in muscle cells for treatment of myotonic dystrophy. In other examples, plasmids expressing shRNA that reduces the mutant Huntington gene (htt) responsible for Huntington's disease can be delivered with neuron specific exosomes. Other target genes include BACE-1 for the treatment of Alzheimer's disease. Some cancer genes may also be targeted with siRNA or shRNAs, such as ras, c-myc and VEGFR-2. Brain targeted siRNA loaded exosomes may be particularly useful in the silencing of BACE-1 in Alzheimer's disease, silencing of alpha-synuclein in Parkinson's disease, silencing of htt in Huntingdon's disease and silencing of neuronal caspase-3 used in the treatment of stroke to reduce ischaemic damage.

Antisense modified oligonucleotides including 2′-O-Me compounds and PNA can be used. For example, such oligonucleotides can be designed to induce exon-skipping for example the mutant dystrophin gene can be delivered to muscle cells for the treatment of Duchenne Muscular Dystrophy, antisense oligonucleotides which inhibit hairpin loops, for example in the treatment of myotonic dystrophy and trans-splicing oligonucleotides, for example for the treatment of spinal muscular atrophy.

As an alternative to loading the exosomes of the invention with genetic material, protein or peptide may be loaded. Any biotherapeutic protein and/or peptide that has utility in the treatment and/or prevention of a condition, disease or disorder may be incorporated into exosomes according to the present invention. In a preferred embodiment the protein or peptide for incorporation into the exosomes is an antibody or an antibody fragment. A single protein or peptide may be incorporated into the exosomes. Alternatively, more than one protein and/or peptide may be incorporated into the exosomes. The more than one proteins and/or peptides may act on the same or different targets to bring about their therapeutic and/or preventative effect.

The proteins and/or peptides to be incorporated into the exosomes may be useful, for example, in the treatment and/or prevention of cancer. Examples of cancers that may be treated using protein or peptide biotherapeutics include leukaemia, colorectal cancer, head and neck cancer, non-Hodgkin lymphoma, breast cancer, ovarian cancer, prostate cancer, gastric cancer, pancreatic cancer, adenocarcinoma and solid tumours. Such biotherapeutic proteins and/or peptides are typically antibodies or fragments thereof, particularly monoclonal antibodies or fragments thereof.

Protein and/or peptide biotherapeutics may also be used in the treatment and/or prevention of autoimmune conditions. Autoimmune conditions arise from an overactive immune response of the body against substances, tissues and organs normally present in the body.

Biotherapeutic proteins and/or peptides loaded into exosomes according to the present invention may also be used to treat and/or prevent other conditions, including cardiovascular disease, haemophilia, sepsis, stroke, muscular dystrophy, including Duchenne muscular dystrophy (DMD), macular degeneration and Alzheimer's Disease.

In a preferred embodiment, the biotherapeutic loaded into exosomes according to the present invention is a therapeutic antibody. In a more preferred embodiment, the therapeutic antibody is selected from a monoclonal antibody for blocking the active site of Bace-1 and b-amyloids; a monoclonal antibody against gliblastoma kinases and an antibody against α4-integrin for the treatment of MS, for example Natalizumab.

In a preferred embodiment the biotherapeutic loaded into exosomes according to the present invention is a peptide. In a more preferred embodiment the peptide is selected from an immune-dominant peptide for eliciting immune responses to viral and/or tumour antigens; a neuroprotective peptide such as a δ-opioid receptor ligand, for example Biphalin; an immune-suppressive peptide for neuroinflammation, for example adrenomedullin and a NBD peptide that binds and inhibits NfKB signalling.

The exogenous genetic material, protein or peptide, or chemotherapeutic agent can be introduced into the exosomes by a number of different techniques. For example, the exosomes may be loaded by electroporation or the use of a transfection reagent. Despite the small size of exosomes, it is still possible to use electroporation to load the exosomes with the exogenous genetic material [WO 2010/119256]. This is surprising in view of the small size of the exosomes compared to cells. Extrapolation of the voltages used for electroporation of cells to take into account the size of the exosomes would suggest that excessively high voltages would be required for electroporation of exosomes. Surprisingly however, it is possible to use electroporation to load exosomes with plasmid DNA and siRNA. Electroporation conditions may vary depending on the charge and size of the cargo. Typical voltages are in the range of 20V/cm to 1000V/cm, such as 20V/cm to 100V/cm with capacitance typically between 25 μF and 250 μF, such as between 25 μF and 125 μF. A voltage in the range of 150 mV to 250 mV, particularly 200 mV, is preferred for loading exosomes of the invention with antibodies.

The exosomes may also be loaded using transfection agents. Despite the small size of the exosomes, conventional transfection agents can be used for transfection of exosomes with genetic material. Preferred transfection reagents for use in accordance with the present invention include cationic liposomes.

Exosomes may also be loaded by transforming or transfecting a host cell with a nucleic acid construct which expressed the genetic material, or biotherapeutic protein or peptide of interest, such that the genetic material, biotherapeutic protein or peptide is taken up into the exosomes as the exosomes are produced in the cell.

Targeting

In accordance with the present invention, the exosomes are targeted to a desired cell type or tissue. This targeting is achieved by expressing on the surface of the exosome a targeting moiety that binds to a cell surface moiety expressed on the surface of the cell to be targeted. In accordance with the present invention, the targeting moiety is a transferrin (Tf) polypeptide or peptide. In a preferred embodiment, the targeting moiety is a Tf peptide. Typically the Tf polypeptide or Tf peptide targeting moiety is expressed as a fusion protein with a transmembrane protein typically expressed on the surface of the exosome.

Tf is a glycoprotein which binds reversibly to iron with high affinity. Human transferrin (Tf-h) consists of a single polypeptide chain with 698 amino acid residues and a molecular weight of approximately 80 kDa. The polypeptide sequence of Tf-h is given in SEQ ID NO: 1. The polynucleotide sequence of the human Tf gene is given in SEQ ID NO: 2. Tf-h binds two Fe3+ ions with a high affinity. Four distinct isoforms of Tf with different iron content coexist in the plasma: (1) apo-transferrin (APOTf, with no iron ions); (2) monoferric transferrin (with iron in the C-terminal domain); (3) monoferric transferrin (with iron in the N-terminal domain) and (4) diferric transferrin (with iron in the two binding sites). In the context of the present invention, Tf encompasses any of these four isotypes, either alone or in combination with each other.

The Tf receptor (TfR) is a disulfide-linked homodimeric glycoprotein that is highly expressed at the cell surface of many tumours. In a preferred embodiment of the invention the moiety present on the cell to be targeted by the exosome of the invention that binds to the Tf polypeptide or Tf peptide targeting moiety expressed on the surface of the exosome is TfR.

In more detail, the exosomes of the invention can be targeted to particular cell types or tissues by expressing on their surface a Tf polypeptide or Tf peptide targeting moiety. Suitable peptides are those which bind to cell surface moieties such as receptors or their ligands found on the cell surface of the cell to be targeted. In a preferred embodiment, the Tf polypeptide or Tf peptide targeting moiety bind to the TfR on the cell surface of the cell to be targeted. Examples of suitable targeting moieties are short peptides and the complete Tf protein, so long as the targeting moiety can be expressed on the surface of the exosome and does not interfere with insertion of the membrane protein into the exosome. Typically the targeting peptide is heterologous to the transmembrane exosomal protein. Peptide targeting moieties may typically be less than 100 amino acids in length, for example less than 50 amino acids in length, less than 30 amino acids in length, to a minimum length of 10, 5 or 4 amino acids.

In a preferred embodiment, the minimum Tf targeting moiety has the amino acid sequence of amino acids 50 to 56 of the Tf protein of SEQ ID NO: 1, i.e. the amino acid sequence PSDGPSV (SEQ ID NO: 4).

In a particularly preferred embodiment, the Tf targeting polypeptide or peptide consists or comprises of the amino acid sequence CRTIGPSVC (SEQ ID NO: 3) and/or amino acids 50 to 56 of the Tf protein of SEQ ID NO: 1, i.e. the amino acid sequence PSDGPSV (SEQ ID NO: 4).

Peptide fragments comprising the amino acid sequence of SEQ ID NOS: 3 or 4 may be used as targeting moieties in the present invention. Such fragments may be 10 amino acids or less in length, 20 amino acids or less in length, 30 amino acids or less in length, 50 amino acids or less in length, 100 amino acids or less in length or more. In a preferred embodiment the fragments are 10 amino acids or less in length. The fragments are preferably fragments of the Tf protein of SEQ ID NO: 1. The full-length Tf protein may also be used as a targeting moiety in the exosomes of the invention.

TfRs are highly expressed on immature erythroid cells, placental tissue and rapidly dividing cells, both normal and malignant. Thus, Tf protein, polypeptide and peptide targeting moieties can be used to target particular tissue types such as for example, or to target a diseased tissue such as a tumour. In a particularly preferred embodiment of the present invention, the exosomes are targeted to tumours.

The Tf targeted exosomes of the present invention may be particularly advantageous for enabling delivery of cargo to all cell types and across species. This is because Tf is highly conserved across species and the TfR is expressed ubiquitously on the surface of many cell types. Thus, for example, a mouse exosome of the present invention may be taken up by human cells and a human exosome of the present invention may be taken up by mouse cells. As such, Tf targeted exosomes may be considered as a universal in vitro transfection reagent. In addition, in vivo Tf targeted exosomes would be ideal for targeting tissues with the elevated TfR expression, such as endothelial cells of the blood brain barrier (BBB), tumour cells and sites of local hypoxia (as in ischemia of the heart or following a stroke).

Thus, potential uses of the Tf targeted exosomes of the present invention include the targeting of genetic and non-genetic cargo across the BBB; the targeting of cancer therapies to tumours, because tumour cells express significantly elevated levels of TfR relative to surrounding tissue; and the targeting of therapies to diseases involving ischemia, such as ischemic heart disease and stroke, where there is also elevated levels of TfR expression.

Expression of a Tf targeting moiety on the surface of an exosome according to the present invention may increase the uptake of said exosome and its cargo by the target cell by at least two-fold, at least three-fold, at least four-fold, at least five-fold, at least ten-fold or at least twenty-fold compared to an untargeted exosome. Expression of a Tf targeting moiety on the surface of an exosome according to the present invention may result in the uptake of the exosome cargo by the target cell at a comparable level to the level of uptake of the same cargo delivered using conventional commercially available transfection reagents such as lipofectamine or RNAi max. Alternatively, expression of a Tf targeting moiety on the surface of an exosome according to the present invention may increase the uptake of the exosome cargo by the target cell compared to the level of uptake of the same cargo delivered using conventional commercially available transfection reagents by at least two-fold, at least three-fold, at least four-fold, at least five-fold, at least ten-fold or at least twenty-fold.

The Tf polypeptide or Tf peptide targeting moiety is expressed on the surface of the exosome by expressing it as a fusion protein with an exosomal transmembrane protein. A number of proteins are known to be associated with exosomes; that is they are incorporated into the exosome as it is formed. The preferred proteins for use in accordance with the present invention are those which are transmembrane proteins. Examples include but are not limited to Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins. In particularly preferred embodiments of the present invention, the transmembrane protein is selected from Lamp-1, Lamp-2, CD13, CD86, Flotillin, Syntaxin-3. In a particularly preferred embodiment the transmembrane protein is Lamp-2. The sequence of Lamp-2 is set out in SEQ ID NO: 5.

The following section relates to general features of all polypeptides of the invention, and in particular to variations, alterations, modifications or derivatisations of amino acid sequence which are included within the polypeptides of the invention. It will be understood that such variations, alterations, modifications or derivatisations of polypeptides as are described herein are subject to the requirement that the polypeptides retain any further required activity or characteristic as may be specified subsequent sections of this disclosure.

Variants of polypeptides of the invention may be defined by particular levels of amino acid identity which are described in more detail in subsequent sections of this disclosure. Amino acid identity may be calculated using any suitable algorithm. For example the PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate homology (for example used on its default settings) (Devereux et at (1984) Nucleic Acids Research 12, 387-395).

It will be understood that variants of polypeptides of the invention also includes substitution variants. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, |
| | | | charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, hydrophobic |

The amino acid sequence of polypeptides for use in the invention may be modified to include non-naturally occurring chemistries or to increase the stability and targeting specificity of the compound. When the polypeptides are produced by synthetic means, such amino acids may be introduced during production. The polypeptides may also be modified following either synthetic or recombinant production.

A number of side chain modifications are known in the art and may be made to the side chains of the polypeptides, subject to the polypeptides retaining any further required activity or characteristic as may be specified herein.

Variant polypeptides as described in this section are those for which the amino acid sequence varies from that in SEQ ID NO: 1 and peptides from SEQ ID NO: 1 or for which the amino acid sequence varies from SEQ ID NOS: 3 and/or 4 and peptides and polypeptides comprising one or both of these sequences, but which retain the ability to bind to the TfR. Alternatively, variant polypeptides as described in this section may be those for which the amino acid sequence varies from that in SEQ ID NO: 5, but which retain the ability to be inserted into the membrane of an exosome.

The variant sequences typically differ by at least 1, 2, 3, 5, 10, 20, 30, 50, 100 or more mutations (which may be substitutions, deletions or insertions of amino acids). For example, from 1 to 100, 2 to 50, 3 to 30 or 5 to 20 amino acid substitutions, deletions or insertions may be made, provided the modified polypeptide is inserted into the membrane of an exosome.

Typically, polypeptides or peptides which are variants of Tf polypeptides or Tf peptides have more than about 50%, 55% or 65% identity, preferably at least 70%, at least 80%, at least 90% and particularly preferably at least 95%, at least 97% or at least 99% identity, with the amino acid sequence of SEQ ID NO: 1, 3 or 4 or the corresponding Tf peptide sequence from SEQ ID NO: 1, 3 or 4.

Typically, polypeptides which are variants of Lamp-2 have more than about 50%, 55% or 65% identity, preferably at least 70%, at least 80%, at least 90% and particularly preferably at least 95%, at least 97% or at least 99% identity, with the amino acid sequence of SEQ ID NO: 5.

The identity of variants of SEQ ID NOS: 1, 3, 4 or 5 may be measured over a region of at least 5, 10, 20, 30, 50, 100, 200, 250, 300, 350 or more contiguous amino acids of the sequence shown in SEQ ID NO: 1, 3, 4 or 5 respectively, or more preferably over the full length of SEQ ID NO: 1, 3, 4 or 5, excluding the signal sequence of SEQ ID NO: 1 or 5.

The Tf peptide targeting moiety used in the invention is typically less than 100 amino acids in length, for example less than 50 amino acids in length, less than 30 amino acids in length, to a minimum length of 10, 5 or 3 amino acids.

The fragment of the Lamp-2 polypeptide used in the invention is typically at least 55 amino acids, 100, 150, 200, or 250 amino acids in length.

The exosomal transmembrane protein is modified to incorporate a Tf targeting moiety. Thus the exosomal transmembrane protein is expressed as a fusion protein comprising the Tf targeting moiety. The Tf targeting moiety is incorporated into the transmembrane protein such that it is positioned in the portion of the transmembrane protein present on the surface of the exosomes. In a preferred aspect of the present invention, the exosomal transmembrane protein is Lamp-2 and the Tf targeting moiety is expressed as a fusion protein, wherein the Tf targeting moiety is present near the N-terminus of Lamp-2 protein for example within 30, or within 20 amino acids of the Lamp-2 N terminal amino acid, not including the signal sequence.

Spacer or linker sequences may be provided between the Tf targeting moiety and the remainder of the transmembrane protein for example to avoid interference from the Tf targeting moiety in the folding of the transmembrane protein.

Linker or spacer sequences are typically 1 to 10 amino acids in length, typically 1 to 8 amino acids in length such as 2, 3 or 4 amino acids in length. Suitable amino acids for incorporation in linkers are alanine, arginine, serine or glycine. Suitable linkers include Ala-Arg and Ser-Gly-Gly.

In a particularly preferred aspect of the present invention, the transmembrane protein is Lamp-2 and the Tf targeting moiety is present at or near the N-terminus of the protein, separated from Lamp-2 with linker sequences.

In the practice of the present invention, the Tf targeting moiety is introduced into the exosome by expressing the fusion protein comprising the Tf targeting moiety and exosomal transmembrane protein within a cell used to produce the exosomes. Expression of this fusion protein in the cell, allows for the fusion protein to be incorporated into the exosome as it is produced from the cell.

For example, a polynucleotide construct such as a DNA plasmid, which expressed the fusion protein is transfected into the cell. Any suitable method can be used for introduction of the polynucleotide construct into the cell. The polynucleotide construct includes suitable promoter sequences so that the encoded fusion protein is expressed in the cell. Signal peptide sequences are also included so that the protein is incorporated into the membrane of the endoplasmic reticulum as it is produced. The membrane protein is then subsequently exported to the exosomal/lysomal compartment before incorporation into the exosome. The signal sequence is typically a signal peptide sequence for an exosomal transmembrane protein. For example the signal peptide sequence is preferably derived from Lamp-2.

Preferred cells for production of exosomes are discussed in more detail above. Typically a preferred cell, such as an immature dendritic cell is transfected with a polynucleotide construct as described above, such that the fusion protein of the invention is expressed in the cell. Exosomes produced by the cell can then be collected. Such exosomes have the fusion protein inserted into the membrane such that the exosomes are targeted to the desired tissue or cell type through the targeting moiety.

Exosomes produced from cells can be collected from the culture medium by any suitable method. Typically a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 μm filter), gradient ultracentrifugation (for example, with sucrose gradient) or a combination of these methods.

In accordance with a preferred aspect of the present invention, the targeted exosomes are loaded with exogenous genetic material. In particular, in accordance with the present invention, exosomes are prepared with a targeting moiety as described herein and then loaded with the desired genetic material for delivery or described above.

In some embodiments of the invention, the exosomes are selected such that they are more likely to target a specific tissue type. For example, exosomes derived from different cells may have natural affinities for specific cell subtypes as required by their physiological function such as the well-established affinity of mature dendritic cell-derived exosomes to T-cells. This affinity may be utilized to assist in the specific delivery of the above-mentioned cargo to a tissue.

Delivery/Administration

The constructs of the invention may be administered by any suitable means. Administration to a human or animal subject may be selected from parenteral, intramuscular, intracerebral, intravascular, (including intravenous), subcutaneous, intranasal, intracardiac, intracerebroventricular, intraperitoneal or transdermal administration. Typically the method of delivery is by injection. Preferably the injection is intramuscular or intravascular (e.g. intravenous). A physician will be able to determine the required route of administration for each particular patient.

The constructs are preferably delivered as a composition. The composition may be formulated for any suitable means of administration, including parenteral, intramuscular, intracerebral, intravascular (including intravenous), intracardiac, intracerebroventricular, intraperitoneal, subcutaneous, intranasal or transdermal administration. Compositions for parenteral administration may include sterile aqueous solutions which may also contain buffers, diluents and other suitable additives. The constructs of the invention may be formulated in a pharmaceutical composition, which may include pharmaceutically acceptable carriers, thickeners, diluents, buffers, preservatives, and other pharmaceutically acceptable carriers or excipients and the like in addition to the exosomes.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

The invention is hereinafter described in more detail with reference to the following Examples.

### EXAMPLES

### Example 1

### Generation of Constructs Encoding a Tf Targeting Moiety

The Tf targeting ligand was attached to the N-terminus after the signal peptide because Lamp2b is a type I membrane protein with an N-terminus predicted to protrude out of the exosome. The TF targeting peptide was inserted into the Lamp2b sequences such that it would be located on the external surface of exosomes, hence conferring targeting capabilities to the exosomes.

The Tf-derived peptide CRTIGPSVC (SEQ ID NO: 3) encoded by the polynucleotide sequence TGTCGTACCATCGGACCAAGTGTTTGT (SEQ ID NO: 7) was inserted into the Lamp2b-expression vector previously described in International Publication No. WO/2010/119256.

The expression vector is based on pEGFP-C1 vector (Clonetech) from which the eGFP gene has been removed. Lamp2b was cloned with cDNA from C2C12 cells and XhoI and BspEI restriction sites were inserted after the signal peptide sequence together with glycine linkers (Ala-Arg-{Targeting Peptide}-Ser-Gly-Gly). The signal peptide of Lamp2b is required for membrane insertion but is cleaved off in the mature protein. The full construct was then cloned downstream of the CMV promoter with NheI and BamHI restriction sites into a pEGFP-C1 vector, removing the eGFP in the process.

The additional sequence added after the signal peptide containing the XhoI and BspEI sites enabled insertion of the TF-coding sequence at the N-terminal part of Lamp2b.

The glycine linkers flanking the Tf targeting peptide prevent the Tf targeting peptide from influencing the folding of the Lamp2b protein. Ultimately, the Tf targeting peptide should be located on the external surface of the exosomes, hence conferring targeting capabilities to the exosomes.

The Tf targeting peptide CRTIGPSVC (SEQ ID NO: 3) encoded by the polynucleotide sequence TGTCGTACCATCGGACCAAGTGTTTGT (SEQ ID NO: 7) was cloned into Lamp2b and transfected into dendritic cells 4 days before exosome purification. A major hindrance to the ability to express targeting ligands on the surface of exosomes is that primary dendritic cells are difficult to transfect and can potentially differentiate after transfection. Infection with viral vectors is not ideal either as dendritic cells are likely to be activated by the virus [30], hence producing immunostimulatory molecules that will be incorporated into the resultant exosomes. Minis Bio's TransIT-LT1 reagent was selected as it appeared to efficiently transduce dendritic cells without significantly activating dendritic cells. Transfection of dendritic cells was performed with 5 μg of pLamp2b-Tf peptide expression vector and 5 μl of TransIT LT1 transfection reagent (Minis Bio) in a 6-well plate with 10⁶ cells on Day 4 after harvesting and isolation of exosomes is done on Day 8.

Murine Lysosome-associated membrane glycoprotein
2 (Lamp 2) isoform 2

Unmodified amino acid sequence (SEQ ID NO: 5)
MCLSPVKGAKLILIFLFLGAVQSNALIVNLTDSKGTCLYAEWEMNFTIT
<-- Signal peptide -->
YETTNQTNKTITIAVPDKATHDGSSCGDDRNSAKIMIQFGFAVSWAVNF
TKEASHYSIHDIVLSYNTSDSTVFPGAVAKGVHTVKNPENFKVPLDVIF
KCNSVLTYNLTPVVQKYWGIHLQAFVQNGTVSKNEQVCEEDQTPTT
VAPIIHTTAPSTTTTLTPTSTPTPTPTPTPTVGNYSIRNGNTTCLLATM
<-- hinge region -->
GLQLNITEEKVPFIFNINPATTNFTGSCQPQSAQLRLNNSQIKYLDFIF
AVKNEKRFYLKEVNVYMYLANGSAFNISNKNLSFWDAPLGSSYMCNKEQ
VLSVSRAFQINTFNLKVQPFNVTKGQYSTAQECSLDDDTILIPIIVGAG
<-- Trans-
LSGLIIVIVIAYLIGRRKTYAGYQTL
membrane -->
Modified amino acid sequence (added sequence
underlined) (SEQ ID NOS: 8 and 9)
MCLSPVKGAKLILIFLFLGAVQSNALIVNLTDSKGTCLYA <peptide
tag>GGAEWEMNFTITYETTNQTNKTITIAVPDKATHDGSSCGDDRNSA
KIMIQFGFAKTITIAVPDKATHDGSSCGDDRNSAKIMIQFGFAVSWAVN
FTKEASHYSIHDIVLSYNTSDSTVFPGAVAKGVHTVKNPENFKVPLDVI
FKCNSVLTYNLTPVVQKYWGIHLQAFVQNGTVSKNEQVCEEDQTPTTVA
PIIHTTAPSTTTTLTPTSTPTPTPTPTPTVGNYSIRNGNTTCLLATMGL
QLNITEEKVPFIFNINPATTNFTGSCQPQSAQLRLNNSQIKYLDFIFAV
KNEKRFYLKEVNVYMYLANGSAFNISNKNLSFWDAPLGSSYMCNKEQVL
SVSRAFQINTFNLKVQPFNVTKGQYSTAQECSLDDDTILIPIIVGAGLS
GLIIVIVIAYLIGRRKTYAGYQTL
Unmodified nucleotide sequence (SEQ ID NO: 6)
atgtgcctctctccggttaaaggcgcaaagctcatcctgatctttctgt
tcctaggagccgttcagtccaatgcattgatagttaatttgacagattc
aaagggtacttgcctttatgcagaatgggagatgaatttcacaataaca
tatgaaactacaaaccaaaccaataaaactataaccattgcagtacctg
acaaggcgacacacgatggaagcagttgtggggatgaccggaatagtgc
caaaataatgatacaatttggattcgctgtctcttgggctgtgaatttt
accaaggaagcatctcattattcaattcatgacatcgtgctttcctaca
acactagtgatagcacagtatttcctggtgctgtagctaaaggagttca
tactgttaaaaatcctgagaatttcaaagttccattggatgtcatcttt
aagtgcaatagtgttttaacttacaacctgactcctgtcgttcagaaat
attggggtattcacctgcaagcttttgtccaaaatggtacagtgagtaa
aaatgaacaagtgtgtgaagaagaccaaactcccaccactgtggcaccc
atcattcacaccactgccccgtcgactacaactacactcactccaactt
caacacccactccaactccaactccaactccaaccgttggaaactacag
cattagaaatggcaatactacctgtctgctggctaccatggggctgcag
ctgaacatcactgaggagaaggtgcctttcatttttaacatcaaccctg
ccacaaccaacttcaccggcagctgtcaacctcaaagtgctcaacttag
gctgaacaacagccaaattaagtatcttgactttatctttgctgtgaaa
aatgaaaaacggttctatctgaaggaagtgaatgtctacatgtatttgg
ctaatggctcagctttcaacatttccaacaagaaccttagcttctggga
tgcccctctgggaagttcttatatgtgcaacaaagagcaggtgctttct
gtgtctagagcgtttcagatcaacacctttaacctaaaggtgcaacctt
ttaatgtgacaaaaggacagtattctacagcccaggagtgttcgctgga
tgatgacaccattctaataccaattatagttggtgctggtctttcaggc
ttgattatcgttatagtgattgcttacctaattggcagaagaaagacct
atgctggatatcagactctgtaa
Modified nucleotide sequence (added sequence in
italics and restrictions sites used for cloning
in underlined) (SEQ ID NO: 10)
atgtgcctctctccggttaaaggcgcaaagctcatcctgatctttctgt
tcctaggagcgttcagtccaatgcattgatagttaatttgacagattca

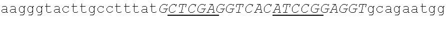

gagatgaatttcacaataacatatgaaactacaaaccaaaccaataaaa
ctataaccattgcagtacctgacaaggcgacacacgatggaagcagttg
tggggatgaccggaatagtgccaaaataatgatacaatttggattcgct
gtctcttgggctgtgaattttaccaaggaagcatctcattattcaattc
atgacatcgtgctttcctacaacactagtgatagcacagtatttcctgg
tgctgtagctaaaggagttcatactgttaaaaatcctgagaatttcaaa
gttccattggatgtcatctttaagtgcaatagtgttttaacttacaacc
tgactcctgtcgttcagaaatattggggtattcacctgcaagcttttgt
ccaaaatggtacagtgagtaaaaatgaacaagtgtgtgaagaagaccaa
actcccaccactgtggcacccatcattcacaccactgccccgtcgacta
caactacactcactccaacttcaacacccactccaactccaactccaac
tccaaccgttggaaactacagcattagaaatggcaatactacctgtctg
ctggctaccatggggctgcagctgaacatcactgaggagaaggtgcctt
tcatttttaacatcaaccctgccacaaccaacttcaccggcagctgtca
acctcaaagtgctcaacttaggctgaacaacagccaaattaagtatctt
gactttatctttgctgtgaaaaatgaaaaacggttctatctgaaggaag
tgaatgtctacatgtatttggctaatggctcagctttcaacatttccaa
caagaaccttagcttctgggatgcccctctgggaagttcttatatgtgc
aacaaagagcaggtgctttctgtgtctagagcgtttcagatcaacacct
ttaacctaaaggtgcaaccttttaatgtgacaaaaggacagtattctac
agcccaggagtgttcgctggatgatgacaccattctaataccaattata
gttggtgctggtctttcaggcttgattatcgttatagtgattgcttacc
taattggcagaagaaagacctatgctggatatcagactctgtaa

### Cell Culture

Dendritic cells were cultured in DC medium (DMEM Glutamax, 10% FBS, non-essential amino acids # and antibiotics). C2C12 cells were cultured in DMEM Glutamax supplemented with 20% FBS and antibiotics.

### Example 2

### Gene Knockdown by siRNA Delivered Using Exosomes Expressing a Tf Targeting Moiety

We aimed to compare the knockdown of a target gene by siRNA delivered using exosomes with Tf targeting moieties on their surface with the knockdown achieved by siRNA delivered using non-targeted exosomes, exosomes targeted by an RVG peptide and siRNA delivered using conventional siRNA tranfection reagent.

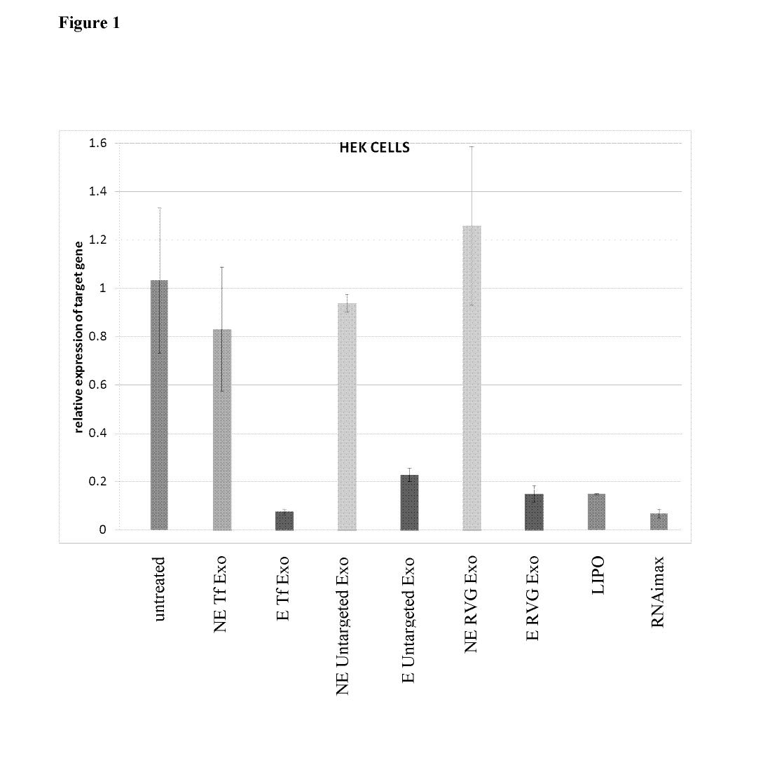

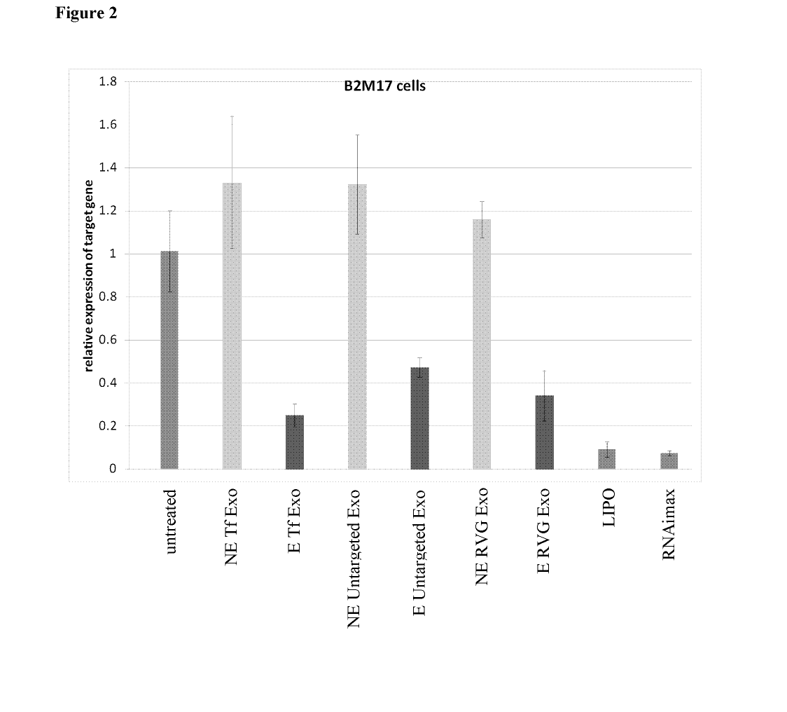

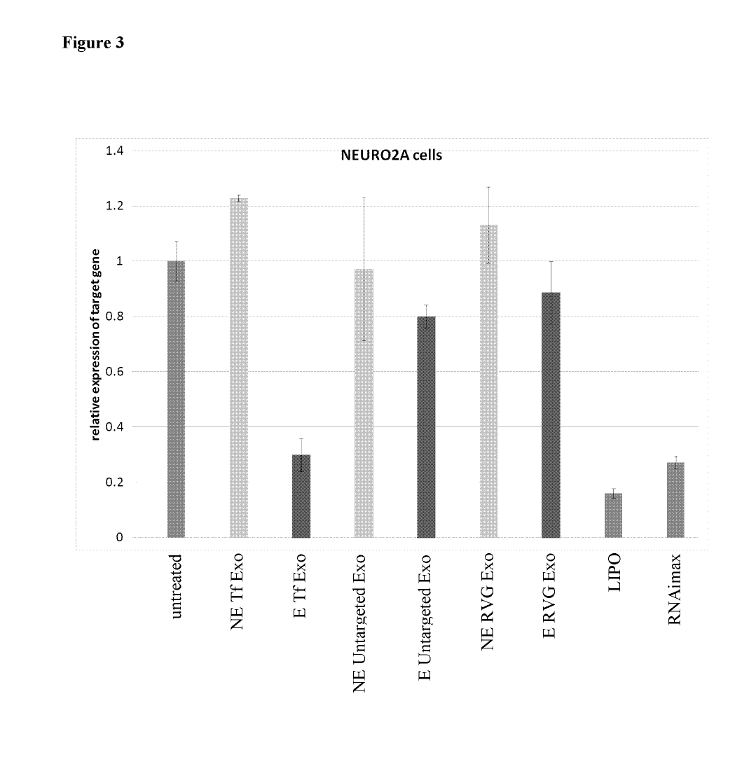

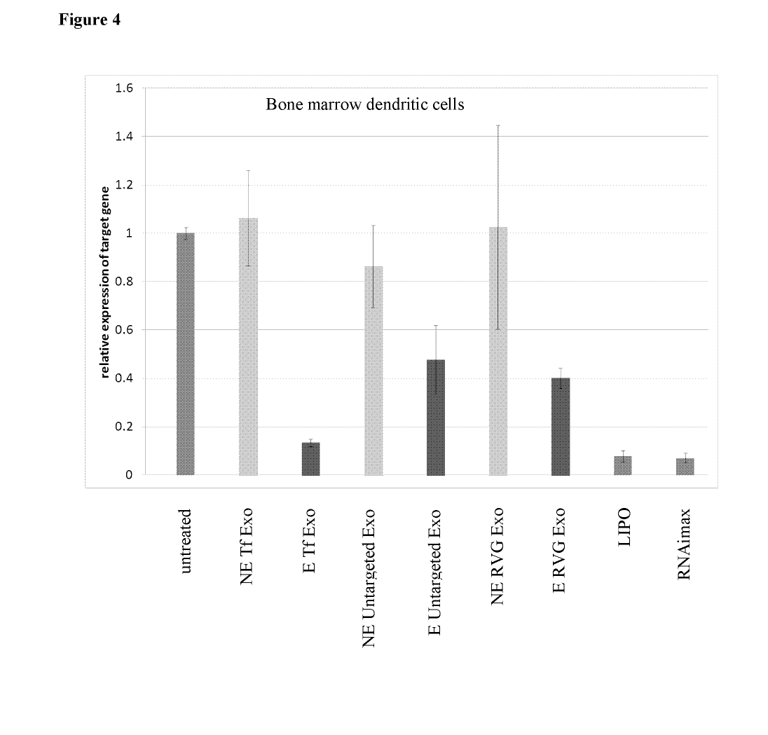

SiRNA for inhibiting GAPDH was delivered to human cell lines (HEK and B2M17) and mouse cells (N2A and bone marrow dendritic cells) using Tf targeted exosomes, RVG targeted exosomes, untargetted exosomes and two commercial siRNA transfection reagents (lipofectamine (LIPO) and RNAi max). Where exosomes were used, these were derived from dendritic cells. For each exosome treatment, a negative control consisiting of the exosomes mixed with the siRNA but not electroporated (NE) was included in addition to the electroporated exosomes (E). As can be seen in FIGS. 1 to 4, Tf-exosomes are better than untargeted and RVG exosomes at reduced GADPH expression in all cell types, and are also comparable in efficiency to the commercial transfection reagents lipofectamine and RNAi max.
¹Smith A J, Schlichtenbrede F C, Tschernutter M, Bainbridge J W, Thrasher A J, Ali R R (2003), AAV-Mediated gene transfer slows photoreceptor loss in the RCS rat model of retinitis pigmentosa, Mol Ther 8(2):188-95.
²Ely A, Naidoo T, Mufamadi S, Crowther C, Arbuthnot P (2008), Expressed anti-HBV primary microRNA shuttles inhibit viral replication efficiently in vitro and in vivo, Mol Ther 16(6):1105-12.

³White M D, Farmer M, Mirabile I, Brandner S, Collinge J, Mallucci G R (2008), Single treatment with RNAi against prion protein rescues early neuronal dysfunction and prolongs survival in mice with prion disease, Proc Natl Acad Sci USA 105(29): 10238-43.
⁴Reynolds A, Leake D, Boese Q, Scaringe S, Marshall W S, Khvorova A (2004), Rational siRNA design for RNA interference, Nat Biotechnol 22(3):326-30.
⁵Takakura Y, Nishikawa M, Yamashita F, Hashida M (2001), Development of gene drug delivery systems based on pharmacokinetic studies, Eur J Pharm Sci 13(1):71-76.
⁶Kawabata K, Takakura Y, Hashida M (1995), The fate of plasmid DNA after intravenous injection in mice: involvement of scavenger receptors in its hepatic uptake, Pharm Res 12(6):825-30.
⁷http:/www.wiley.co.uk/genmed/clinical/ Retrieved on 9 Sep. 2008.
⁸Lowenstein P R, Mandel R J, Xiong W D, Kroeger K, Castro M G (2007), Immune responses to adenovirus and adeno-associated vectors used for gene therapy of brain diseases: the role of immunological synapses in understanding the cell biology of neuroimmune interactions, Curr Gene Ther 7(5):347-60.
⁹Alexander I E, Cunningham S C, Logan G J, Christodoulou J (2008), Potential of AAV vectors in the treatment of metabolic disease,Gene Ther.; 15(11):831-9.
¹⁰Hasbrouck N C, High K A (2008), AAV-mediated gene transfer for the treatment of hemophilia B: problems and prospects, Gene Ther 15(11):870-5.
¹¹Daniel R, Smith J A (2008), Integration site selection by retroviral vectors: molecular mechanism and clinical consequences, Hum Gene Ther 19(6):557-68.
¹²Zhang J S, Liu F, Huang L (2005), Implications of pharmacokinetic behavior of lipoplex for its inflammatory toxicity, Adv Drug Deliv Rev 57(5):689-98.
¹³Ishida T, Masuda K, Ichikawa T, Ichihara M, Irimura K, Kiwada H (2005), Accelerated clearance of a second injection of PEGylated liposomes in mice, Int J Pharm 255:167-74.
¹⁴Ishida T, Ichihara M, Wang X, Yamamoto K, Kimura J, Majima E, Kiwada H. (2006), Injection of PEGylated liposomes in rats elicits PEG-specific IgM, which is responsible for rapid elimination of a second dose of PEGylated liposomes, J Control Release 112:15-25.
¹⁵Ishida T, Ichihara M, Wang X, Yamamoto K, Kimura J, Majima E, Kiwada H (2006), Injection of PEGylated liposomes in rats elicits PEG-specific IgM, which is responsible for rapid elimination of a second dose of PEGylated liposomes, J Control Release 112:15-25.
¹⁶Jiang H, Couto L B, Patarroyo-White S, Liu T, Nagy D, Vargas J A, et al (2006), Effects of transient immunosuppression on adenoassociated, virus-mediated, liver-directed gene transfer in rhesus macaques and implications for human gene therapy, Blood 108(10):3321-8.
¹⁷Wang Z, Kuhr C S, Allen J M, Blankinship M, Gregorevic P, Chamberlain J S, et al (2007), Sustained AAV-mediated dystrophin expression in a canine model of Duchenne muscular dystrophy with a brief course of immunosuppression, Mol Ther 15(6):1160-6.
¹⁸Li J Z, Li H, Hankins G R, Dunford B, Helm G A (2005), Local immunomodulation with CD4 and CD8 antibodies, but not cyclosporine A, improves osteogenesis induced by ADhBMP9 gene therapy, Gene Ther 12(16):1235-41.
¹⁹Zaiss A K, Muruve D A (2005), Immune responses to adeno-associated viral vectors, Curr Gene Ther 5(3):323-31.
²⁰Taylor N, Uribe L, Smith S, Jahn T, Kohn D B, Weinberg K (1996), Correction of interleukin-2 receptor function in X-SCID lymphoblastoid cells by retrovirally mediated transfer of the gamma-c gene, Blood 87(8):3103-7.
²¹Oh Y K, Sohn T, Park J S, Kang M J, Choi H G, Kim J A, et al (2004), Enhanced mucosal and systemic immunogenicity of human papillomavirus-like particles encapsidating interleukin-2 gene adjuvant, Virology 328(2):266-73.
²²Williams D A (2007), RAC reviews serious adverse event associated with AAV therapy trial, Mol Ther 15(12):2053-4.
²³Raposo G, Nijman H W, Stoorvogel W, Liejendekker R, Harding C V, Melief D J Geuze H J (1996), B lymphocytes secrete antigen-presenting vesicles, J Exp Med 183:1161-1172.
²⁴Zitvogel L, Regnault A, Lozier A, Wolfers J, Flament C, Tenza D, Ricciardi-Castagnoli P, Raposo G, Amigorena S (1998), Eradication of established murine tumors using a novel cell-free vaccine: dendritic cell-derived exosomes, Nat Med 4:594-600.
²⁵Hao S, Moyana T, Xiang J (2007), Review: cancer immunotherapy by exosome-based vaccines, Cancer Biother Radiopharm 22:692-703.
²⁶Valadi H, Ekström K, Bossios A, Sjöstrand M, Lee J J, Lötvall J O (2007), Exosome-mediated transfer of mRNAs and microRNAs is a novel mechanism of genetic exchange between cells, Nat Cell Biol 9(6):654-9.
²⁷Skog J, Würdinger T, van Rijn S, Meijer D H, Gainche L, Sena-Esteves M, Curry W T Jr, Carter B S, Krichevsky A M, Breakefield X O (2008), Glioblastoma microvesicles transport RNA and proteins that promote tumour growth and provide diagnostic biomarkers, Nat Cell Biol 10(12):1470-6.
²⁸WO 2010/119256.

1. A composition comprising an exosome, wherein the exosome comprises a targeting moiety expressed on the surface of the exosome, and said targeting moiety is a transferrin (Tf) polypeptide or peptide.

2. A composition according to claim 1, wherein the Tf polypeptide comprises a polypeptide sequence having at least 80% sequence identity to SEQ ID NO: 1 or the Tf peptide is from said polypeptide.

3. A composition according to claim 1, wherein the Tf peptide comprises at least 10 contiguous amino acid residues from the polypeptide sequence of SEQ ID NO: 1.

4. A composition according to claim 1, wherein the peptide is selected from: CRTIGPSVC (SEQ ID NO: 3) or PSDGPSV (SEQ ID NO: 4).

5. A composition according to claim 1, wherein the targeting moiety comprises a peptide which binds to a moiety present on the cell to be targeted.

6. A composition according to claim 5, wherein the moiety present on the cell to be targeted is a transferrin receptor (TfR).

7. A composition according to claim 5, wherein the exosome comprises an exosomal transmembrane protein which has been modified to incorporate the peptide targeting moiety.

8. A composition according to claim 7, wherein the exosomal transmembrane protein is selected from Lamp-1, Lamp-2, CD13, CD86, Flotillin, Syntaxin-3.

9. A composition according to claim 8, wherein the exosomal transmembrane protein is Lamp-2b.

10. A composition according to claim 9, wherein the targeting moiety is present at or near the N-terminus of Lamp-2b and is separated from Lamp-2b with linker sequences.

11. A composition according to claim 1, wherein the exosome is derived from dendritic cells.

12. A composition according to claim 1, wherein the exosome is loaded with exogenous genetic material, protein and/or peptide, or a chemotherapeutic agent.

13. A composition according to claim 12, wherein the exogenous genetic material is a DNA plasmid encoding a therapeutic protein or an immunogen.

14. A composition according to claim 12, wherein the exogenous genetic material is an siRNA.

15. A composition according to claim 13, wherein the plasmid encodes a therapeutic protein for use in a method of gene therapy.

16. A composition according to claim 13, wherein the plasmid encodes an immunogen, for use in a method of generating an immune response to the immunogen.

17. A composition according to claim 12, wherein the exosome is derived from an immature dendritic cell, for use in a method of delivering the genetic material, protein and/or peptide or chemotherapeutic agent in vivo.

18. A method of producing an exosome comprising a Tf polypeptide or Tf peptide targeting moiety expressed on the surface of the exosome comprising expressing a fusion protein comprising the targeting moiety and an exosomal transmembrane protein within a cell used to produce exosomes, wherein the expressed fusion protein is incorporated into the exosome as it is produced by the cell.

19. A method according to claim 18, wherein a polynucleotide construct encoding for the fusion protein, said polynucleotide construct further comprising a promoter and a signal peptide sequence, is transfected into the cell.

20. A method according to claim 18, wherein the cell is a dendritic cell.

21. A polypeptide comprising an exosomal transmembrane protein, and a heterologous targeting Tf polypeptide or Tf peptide, wherein the targeting Tf polypeptide or Tf peptide binds to a Tf receptor (TfR) present on the surface of the cell to be targeted, and wherein when the polypeptide is present in an exosome, the targeting Tf polypeptide or Tf peptide is present on the surface of the exosome.

22. A polynucleotide construct encoding a polypeptide as defined in claim 22, wherein the construct comprises polynucleotide encoding a 5′ exosomal transmembrane signal sequence operatively linked to polynucleotide encoding the polypeptide of the invention.

23. A polynucleotide construct according to claim 22, wherein the exosomal transmembrane signal sequence is an endoplasmic reticulum-targeting signal peptide.

24. A method of targeting an exosome to a selected tissue or cell type comprising transfecting a host cell with a polynucleotide construct as defined in claim 22, expressing the construct in the host cell, and obtaining exosomes from the host cell in which the construct has been expressed.

25. A method of gene silencing in a non human animal model, comprising administering to the animal an exosome according to claim 14, wherein the siRNA is directed against the gene to be silenced.


SEQUENCE LISTING (10 sequences)

SEQ ID NO: 1 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - DNA, 2347 bp, Homo sapiens
  misc_feature (44)..(44): n is a, c, g, or t
acggcgagcctcatcctccgggtgcggcgctgagcagcgagtcncgactgtgctcgctgctcagcgccgcacccggaggatgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcaggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaaaatctcagaggtagggctgccaccaaggtgaagatgggaacgcagatgatccatgagtttgccctggtttcactggcccaagtggtttgtgctaaccacgtctgtcttcacagctctgtgttgccatgtgtgctgaacaaaaaataaaaattattattgattttaaaaaaa

SEQ ID NO: 3 - protein, 9 aa, Artificial Sequence
  Tf targeting peptide comprising GPSV
CRTIGPSVC

SEQ ID NO: 4 - protein, 7 aa, Homo sapiens
PSDGPSV

SEQ ID NO: 5 - protein, 415 aa, Mus musculus
MCLSPVKGAKLILIFLFLGAVQSNALIVNLTDSKGTCLYAEWEMNFTITYETTNQTNKTITIAVPDKATHDGSSCGDDRNSAKIMIQFGFAVSWAVNFTKEASHYSIHDIVLSYNTSDSTVFPGAVAKGVHTVKNPENFKVPLDVIFKCNSVLTYNLTPVVQKYWGIHLQAFVQNGTVSKNEQVCEEDQTPTTVAPIIHTTAPSTTTTLTPTSTPTPTPTPTPTVGNYSIRNGNTTCLLATMGLQLNITEEKVPFIFNINPATTNFTGSCQPQSAQLRLNNSQIKYLDFIFAVKNEKRFYLKEVNVYMYLANGSAFNISNKNLSFWDAPLGSSYMCNKEQVLSVSRAFQINTFNLKVQPFNVTKGQYSTAQECSLDDDTILIPIIVGAGLSGLIIVIVIAYLIGRRKTYAGYQTL

SEQ ID NO: 6 - DNA, 1248 bp, Mus musculus
atgtgcctctctccggttaaaggcgcaaagctcatcctgatctttctgttcctaggagccgttcagtccaatgcattgatagttaatttgacagattcaaagggtacttgcctttatgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagcccaggagtgttcgctggatgatgacaccattctaataccaattatagttggtgctggtctttcaggcttgattatcgttatagtgattgcttacctaattggcagaagaaagacctatgctggatatcagactctgtaa

SEQ ID NO: 7 - DNA, 27 bp, Artificial Sequence
  sequence encoding generated Tf targeting peptide
tgtcgtaccatcggaccaagtgtttgt

SEQ ID NO: 8 - protein, 40 aa, Artificial Sequence
  Fragment of modified amino acid sequence of murine Lamp 2 isoform 2 N-terminal to peptide tag
MCLSPVKGAKLILIFLFLGAVQSNALIVNLTDSKGTCLYA

SEQ ID NO: 9 - protein, 378 aa, Artificial Sequence
  Fragment of modified amino acid sequence of murine Lamp 2 isoform 2 C-terminal to peptide tag
GGAEWEMNFTITYETTNQTNKTITIAVPDKATHDGSSCGDDRNSAKIMIQFGFAVSWAVNFTKEASHYSIHDIVLSYNTSDSTVFPGAVAKGVHTVKNPENFKVPLDVIFKCNSVLTYNLTPVVQKYWGIHLQAFVQNGTVSKNEQVCEEDQTPTTVAPIIHTTAPSTTTTLTPTSTPTPTPTPTPTVGNYSIRNGNTTCLLATMGLQLNITEEKVPFIFNINPATTNFTGSCQPQSAQLRLNNSQIKYLDFIFAVKNEKRFYLKEVNVYMYLANGSAFNISNKNLSFWDAPLGSSYMCNKEQVLSVSRAFQINTFNLKVQPFNVTKGQYSTAQECSLDDDTILIPIIVGAGLSGLIIVIVIAYLIGRRKTYAGYQTL

SEQ ID NO: 10 - DNA, 1270 bp, Artificial Sequence
  Modified nucleotide sequence of murine Lamp 2 isoform 2
atgtgcctctctccggttaaaggcgcaaagctcatcctgatctttctgttcctaggagccgttcagtccaatgcattgatagttaatttgacagattcaaagggtacttgcctttatgctcgaggtcacatccggaggtgcagaatgggagatgaatttcacaataacatatgaaactacaaaccaaaccaataaaactataaccattgcagtacctgacaaggcgacacacgatggaagcagttgtggggatgaccggaatagtgccaaaataatgatacaatttggattcgctgtctcttgggctgtgaattttaccaaggaagcatctcattattcaattcatgacatcgtgctttcctacaacactagtgatagcacagtatttcctggtgctgtagctaaaggagttcatactgttaaaaatcctgagaatttcaaagttccattggatgtcatctttaagtgcaatagtgttttaacttacaacctgactcctgtcgttcagaaatattggggtattcacctgcaagcttttgtccaaaatggtacagtgagtaaaaatgaacaagtgtgtgaagaagaccaaactcccaccactgtggcacccatcattcacaccactgccccgtcgactacaactacactcactccaacttcaacacccactccaactccaactccaactccaaccgttggaaactacagcattagaaatggcaatactacctgtctgctggctaccatggggctgcagctgaacatcactgaggagaaggtgcctttcatttttaacatcaaccctgccacaaccaacttcaccggcagctgtcaacctcaaagtgctcaacttaggctgaacaacagccaaattaagtatcttgactttatctttgctgtgaaaaatgaaaaacggttctatctgaaggaagtgaatgtctacatgtatttggctaatggctcagctttcaacatttccaacaagaaccttagcttctgggatgcccctctgggaagttcttatatgtgcaacaaagagcaggtgctttctgtgtctagagcgtttcagatcaacacctttaacctaaaggtgcaaccttttaatgtgacaaaaggacagtattctacagcccaggagtgttcgctggatgatgacaccattctaataccaattatagttggtgctggtctttcaggcttgattatcgttatagtgattgcttacctaattggcagaagaaagacctatgctggatatcagactctgtaa